(19) United States
(12) Patent Application Publication
(10) Pub. No.: US 2018/0252699 A1
(43) Pub. Date: Sep. 6, 2018

(54) BREATHALYZER METROLOGICAL BENCH

(71) Applicant: Muc Dang, Toronto (CA)

(72) Inventor: Muc Dang, Toronto (CA)

(21) Appl. No.: 15/718,906

(22) Filed: Sep. 28, 2017

Related U.S. Application Data
(60) Provisional application No. 62/400,872, filed on Sep. 28, 2016.

(51) Int. Cl. G01N 33/497; G01N 33/00

(52) U.S. Cl. CPC G01N 33/4972; A61B 5/4845; G01N 33/0008

(57) ABSTRACT

A metrological bench is for calibrating a breath alcohol tester and for this purpose is adapted to carry out a method involving delivering to the tester a sample of gas that varies in terms of ethanol concentration, CO₂ concentration, flow rate, pressure and temperature in a manner equivalent to the variances exhibited by a human.

### CROSS-REFERENCE TO RELATED APPLICATION

This application claims the benefit of U.S. Provisional Patent Application Ser. No. 62/400,872, filed Sep. 28, 2016.

### BACKGROUND OF THE INVENTION

### 1. Field of the Invention

The invention relates to the field of alcohol breath tester calibration.

### 2. Prior Art

It is known to calibrate breath alcohol testers using devices that simulate human breath with respect to humidity, time, CO₂ and temperature.

### SUMMARY OF THE INVENTION

Forming one aspect of the invention is a method for calibrating a breath alcohol tester, the method comprising: delivering to the tester a sample of gas that varies in terms of ethanol concentration, CO₂ concentration, flow rate, pressure and temperature in a manner equivalent to the variances exhibited by a human.

Forming another aspect of the invention is apparatus for use with a breath tester and adapted to deliver to the tester a sample of gas that varies in terms of ethanol concentration, CO₂ concentration, flow rate, pressure and temperature in a manner equivalent to the variances exhibited by a human.

Other advantages, features and characteristics of the invention will become apparent upon a review of the detailed description and the appended drawings, the latter being briefly described hereinbelow.

### BRIEF DESCRIPTION OF THE DRAWINGS

FIG. 1 is a perspective view of an exemplary embodiment of the bench;

FIG. 2 is a perspective view of a portion of the bench of FIG. 1;

FIG. 3 is a view of the structure of FIG. 2 from another vantage point;

FIG. 4 is a perspective view of another portion of the bench of FIG. 1;

FIG. 5 is a perspective view of another portion of the bench of FIG. 1;

FIG. 6 is a perspective view of another portion of the bench of FIG. 1;

FIG. 7A is a view of the structure of FIG. 1 from another vantage point;

FIG. 7B is a view of the structure of FIG. 1 from yet another vantage point;

FIG. 8 shows the flow control and pneumatic circuit of the bench of FIG. 1;

FIG. 9A is a view of a portion of a software interface displayed in use of the bench;

FIG. 9B is a view of a portion of a software interface that is displayed in use of the bench along with the interface of FIG. 9A;

FIG. 10A is a view of another software interface displayed in use of the bench;

FIG. 10B is a view of a portion of a software interface that is displayed in use of the bench along with the interface of FIG. 9A;

FIG. 11 is a schematic view of the flow circuit;

FIG. 12 is a view similar to FIG. 11 showing a standby mode of the bench;

FIG. 13 is a view similar to FIG. 11 showing mixture generation;

FIG. 14 is a view similar to FIG. 11 showing sampling;

FIG. 15 is a view similar to FIG. 11 showing the bench waiting for breath;

FIG. 16 is a view similar to FIG. 11 showing breath;

FIG. 17 is a view similar to FIG. 11 showing calibration of the bench;

FIG. 18 is a simplified view of a multiplexer portion of the bench;

FIG. 19 is a view of a portion of the panel; and

FIG. 20 is a view similar to FIG. 19 showing the bench in use.

### DETAILED DESCRIPTION OF THE PREFERRED EMBODIMENTS

The embodiments discussed herein are merely illustrative of specific manners in which to make and use the invention and are not to be interpreted as limiting the scope.

While the invention has been described with a certain degree of particularity, it is to be noted that many modifications may be made in the details of the invention's construction and the arrangement of its components without departing from the scope of this disclosure. It is understood that the invention is not limited to the embodiments set forth herein for purposes of exemplification.

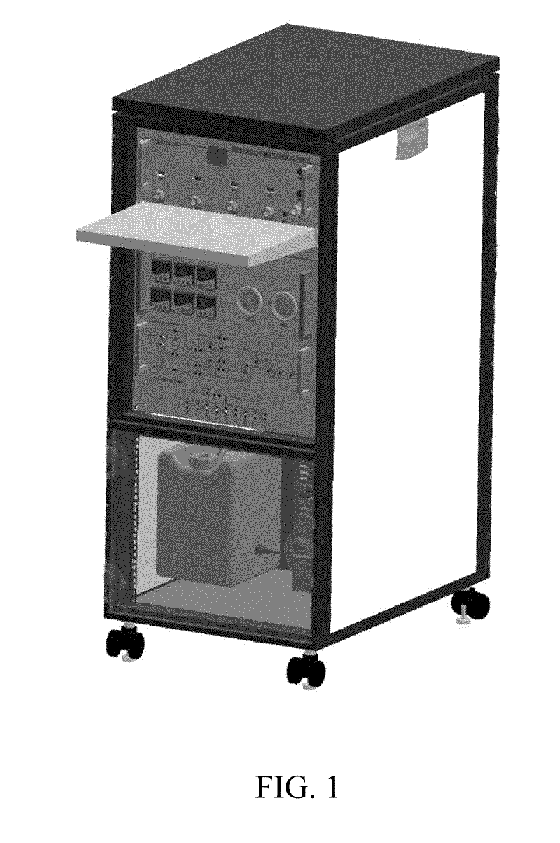

The Breathalyzer Metrological Bench shown in FIG. 1 is dedicated to repeatedly simulate breath. For each breath, the flow rate, volume and ethanol concentration are accurately determined. These breaths have the same characteristics as human breath (humidity, time, CO₂, temperature alveolar volume and “dead volume”). The bench also allows the possibility to work with dry gas. The bench is compatible with tests described in OIML R 126 ed 1998 and ed 2012.

### Construction

The bench has several main parts.

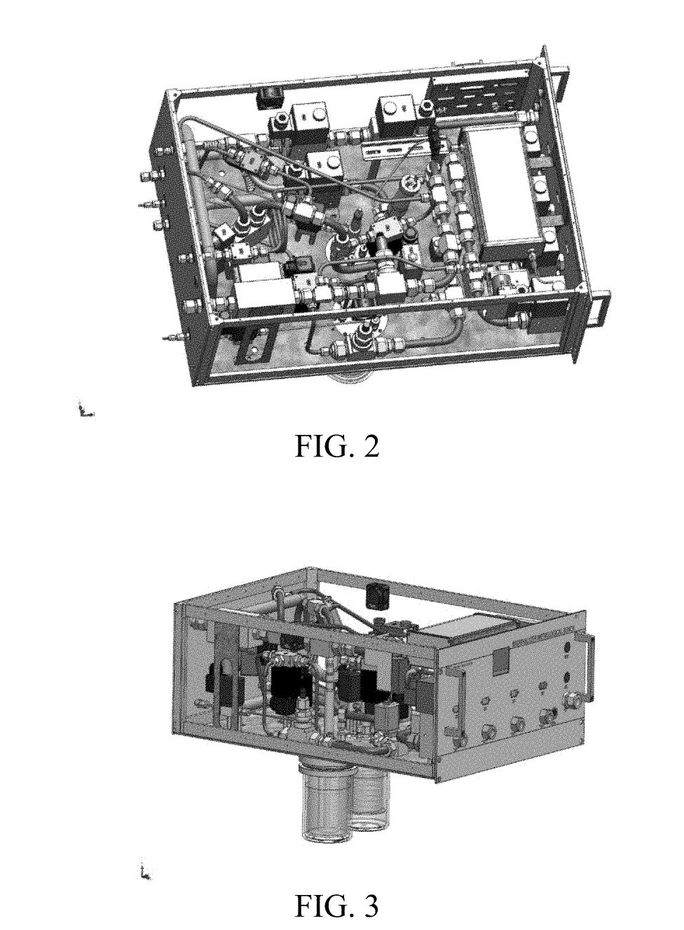

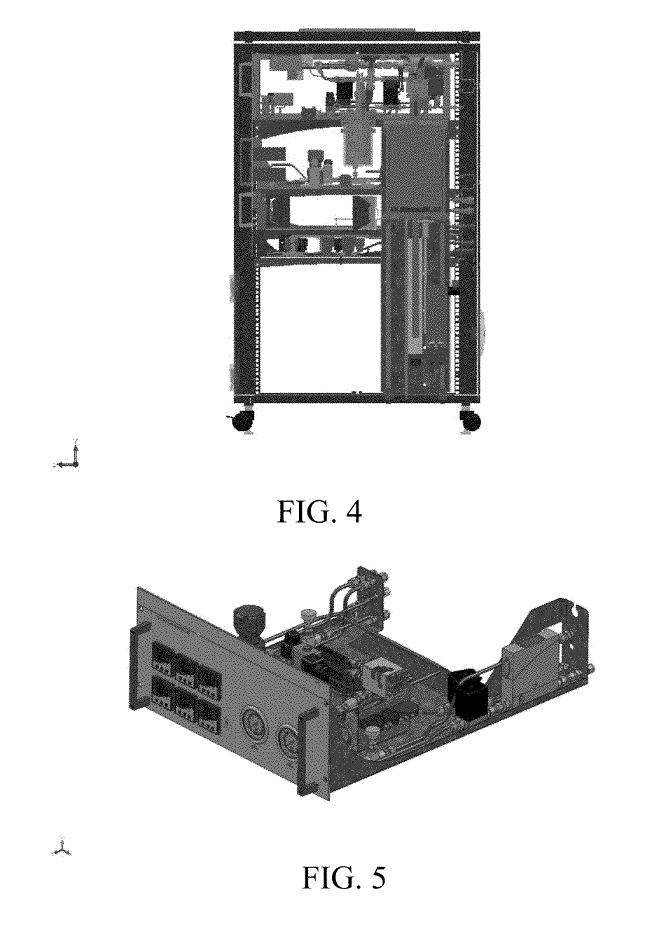

As shown in FIGS. 2-4, two enclosures regulated in temperature; one containing the “artificial lung” and the other containing the system to reproduce the physiological characteristics of human breath (temperature, dead volume, respiration cycle, . . . ).

One rack, shown in FIG. 5, to create and stabilize the gas mixture (CO₂, water, air, ethanol and eventual interferent). A system of nozzles, pressure regulators and mass flow controllers give the flexibility to change concentrations and ensure high stability of the system. Ethanol gas is obtained by bubbling inside a tank which is heated and controlled at 34° C. and contains pure ethanol. Humidity is also obtained by bubbling inside a tank which is heated and controlled at 34° C.; however, it contains pure water. In both cases, levels are maintained constant by peristaltic pumps controlled by analogical level sensors. Measurement cells with infrared systems allow the control and monitoring of CO₂ and ethanol concentration. Humidity is monitored by a capacitive probe and temperature with Pt 100 sensors.

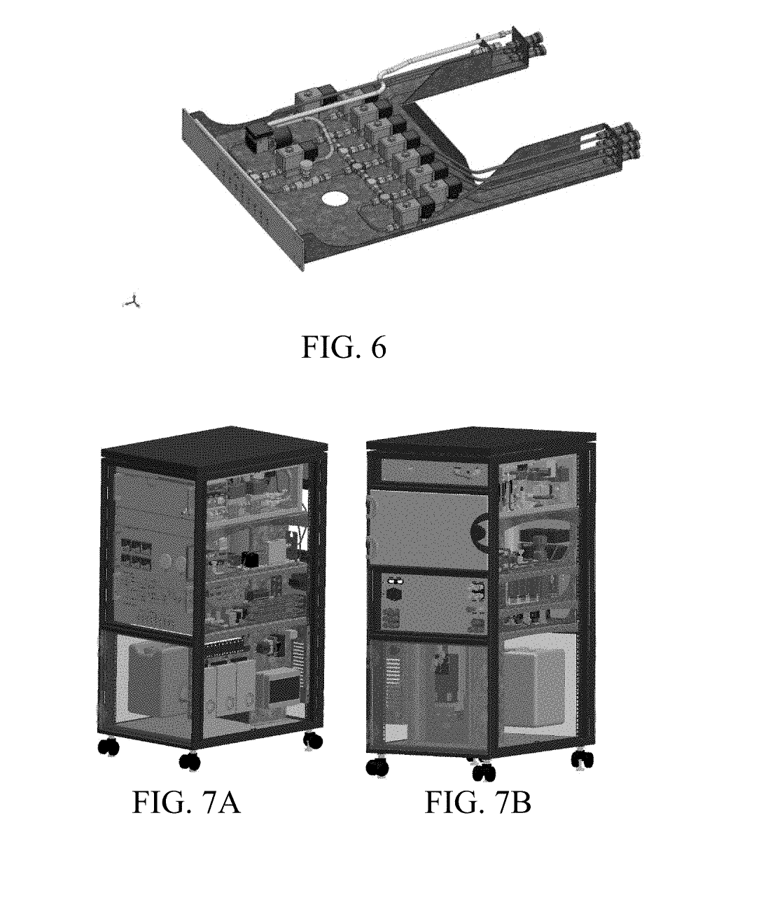

The bench reference is an embedded NDIR analyzer as shown in FIG. 6 with one or two wavelengths centered on ethanol IR spectrum peaks. This reference should be calibrated each month to prevent any drift and ensure the uncertainties of measurement system. It could be calibrated with dry gas inside a cylinder or wet gas by bubbling in hydroalcoholic solutions and applying a vapor pressure formula (as Dubowski or Harger). There is a slight difference between the two, about 1 to 3%, and it is responsibility of each country to determine what will be the reference.

In the case of dry gas, the bench offers a system with 6 to 9 electrovalves connected to a circuit equipped with a vacuum pump that allows to automatically perform a full calibration of internal reference with 6 to 9 different concentrations (range depends on concentrations chosen). Each valve used should be connected to external cylinder (reference mixture) equipped with the pressure regulator.

In the case of wet connection, the bench is connected with heated tubes to an additional external device constituted with flasks regulated in temperatures (liquid and air independently) and equipped with individual temperature sensors. The reference in this case is the concentration of the solutions and temperature measured (please refer to uncertainties calculation document). It is possible for the laboratory to provide its own system of wet gas generation and connect it to the bench with the heated tube (by default ¼″ connection).

The last part is an electronic system. The entire bench is controlled by two independent PLCs (programmable logical controller) with analogical and digital modules. It is possible to monitor and set all parameters by connecting a external PC (with RS485 (or Bluetooth, if given the option) communication).

The various parts are mounted and connected in a 19″ cell width cabinet (height: 130 cm, width: 56 cm, depth: 82 cm). Bench can be powered in 220V/110V and 50/60 H, as shown in FIGS. 7A and 7B.

### Function

Temperature
A not regulated zone: This contains all the temperature-sensitive elements, especially the electronic cards, (PLC, analog/digital modules), the piston motor and mass flow controller, at an ambient temperature. As this is not regulated, it is recommended that the ambient temperature must not be too high (must be <30° C.) or be operating in an air conditioned room.
A 35.5° C. regulated zone: For a wet calibration, it is the temperature of the external calibration device. It is also the temperature of the flexible heated hose and the lung. The regulation is performed to 0.1° C. by a regulator following a PID algorithm with adjustable parameters. A display on the front panel provides the different temperatures.
A 34° C. regulated zone: This concerns the pneumatic circuit outside the regulated enclosure, mainly the connection to the instruments to maintain human breath temperature and avoid any cool spots in case of wet gas (saturated in humidity). Temperature of the two liquid tanks (ethanol and water) is also used to generate moisture and ethanol concentration. The temperature is monitored with a precision of 0.2° C.

### Gas Mixture

The mixture is composed of three main constituents: air, CO₂, and ethanol (or/and eventually an interfering component) and water in case of wet gas. The air must be, if it comes from the general supply system, of excellent quality (dry, without oil and dust). If it comes from a bottle, the minimum quality required is 99.99%. For the CO₂, the minimum quality required is 99.9%. Ethanol is monitored directly by internal reference so its purity is not taken into consideration in the calculation; however, a minimum purity of 99.9% is advised.

Uncertainties of the bench will directly be linked to uncertainties of the dry mixture or the hydro alcoholic solution used to calibrate internal reference (please refer to the uncertainties calculation document). Maximum uncertainties should be 2%.

The air, at the entrance, is divided into three parts: one to ensure the air zero reference, another one to generate ethanol gas by bubbling inside the ethanol tank and the last one constitutes the principal flux, the carrier gas which will be constituted in the mixture. (Note: with the three-way valve system, it is possible to switch the carrier gas to another gas that contains interferent (like acetone or CO).

The ethanol concentration is related to the saturated vapor pressure at a constant temperature. A mass flow controller (range 0 to 150 ml/min.) allows the control of the ethanol flow rate to change the ethanol concentration (with a step <1 μg/l).

For the optimal use of the bench (compromise between speed and stability), it is advised to adjust and fix the carrier gas flow rate to 15 l/min (relative pressure: 2.5 bars) so it will be the continuous flow needed to work with the bench.

The ethanol-saturated air is the last constituent injected in the mixture. The air of the principal flux is mixed with CO₂ and saturated with water (humidity >95%) in a humidificator (34° C. regulated water tank) in case of wet gases, before it is mixed with ethanol. The CO₂ flow rate is normally adjusted to obtain a concentration of 5.0% but can be modified for a particular test, for example, at 10%.

A specific circuit allows to inject interferent in the mixture through a plug system.

### Flow Control and Pneumatic Circuit

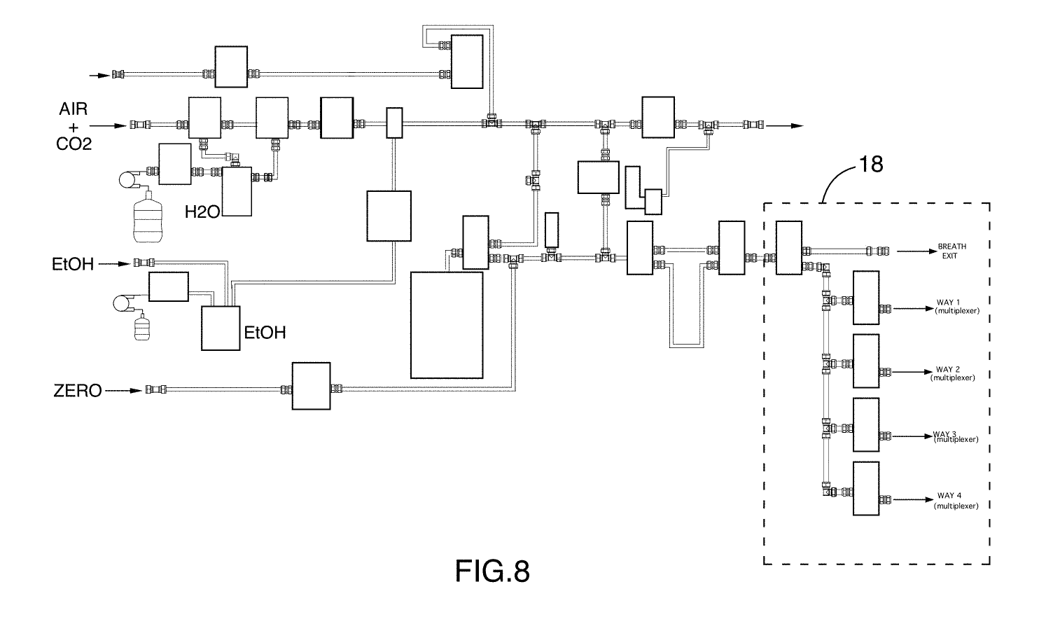

The flow control and pneumatic circuit is shown in FIG. 8. With reference to FIG. 8, it will be understood:

The inspiration/expiration flows are controlled with a piston connected to a three-way valve. This piston, controlled by a brushless motor with a resolver, allows to obtain accurate volumes and times on motion (precision of 1 ml and 0.1 seconds) and to control the flow of inspiration and breath. When the piston is going down (inspiration), the valves are connected at the carrier gas flow and the “lung” takes the necessary volume (the flow rate must be greater than the inspiration flow). After pressure equilibration, the “lung” is ready to breath. The valve is then connected to exit tube (for instruments connection). During all the breaths, the ethanol concentration, the CO₂ concentration, the flow rate, the pressure and the temperature are monitored by the PLC.

Two three lines electrovalves allow to change the “dead volume” (simulation of mouth and respiratory track), modifying then, the time of ethanol concentration plateau at the end of the breath.

Zero air, beyond the breath periods, ensures the purge of different circuits in order to ensure that there is no residual alcohol between two cycles.

### Software

The bench has several pieces of associated software, as noted below.

### PLC Software

It controls in real time the different parts of the bench such as the power on/off valves and pumps, and provides input/output of different parameters (alarms, analog and digital information) and controls regulation.

### Windows Software

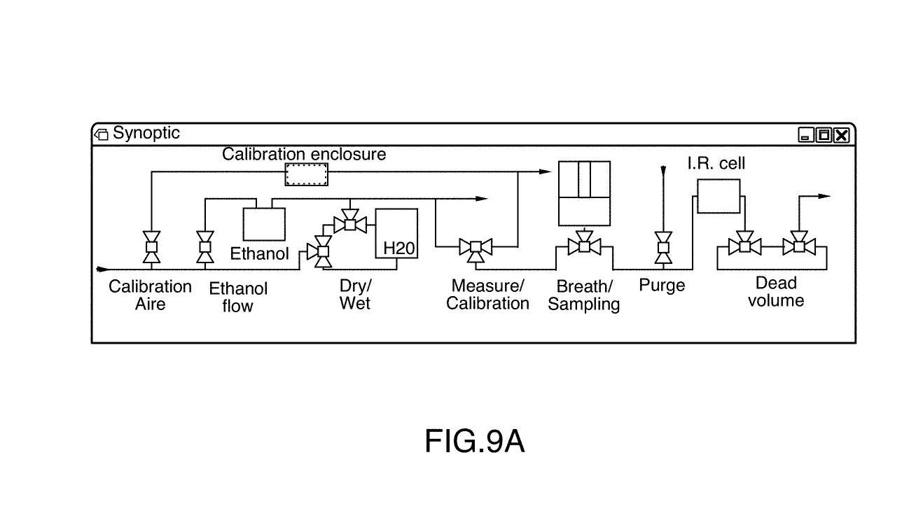

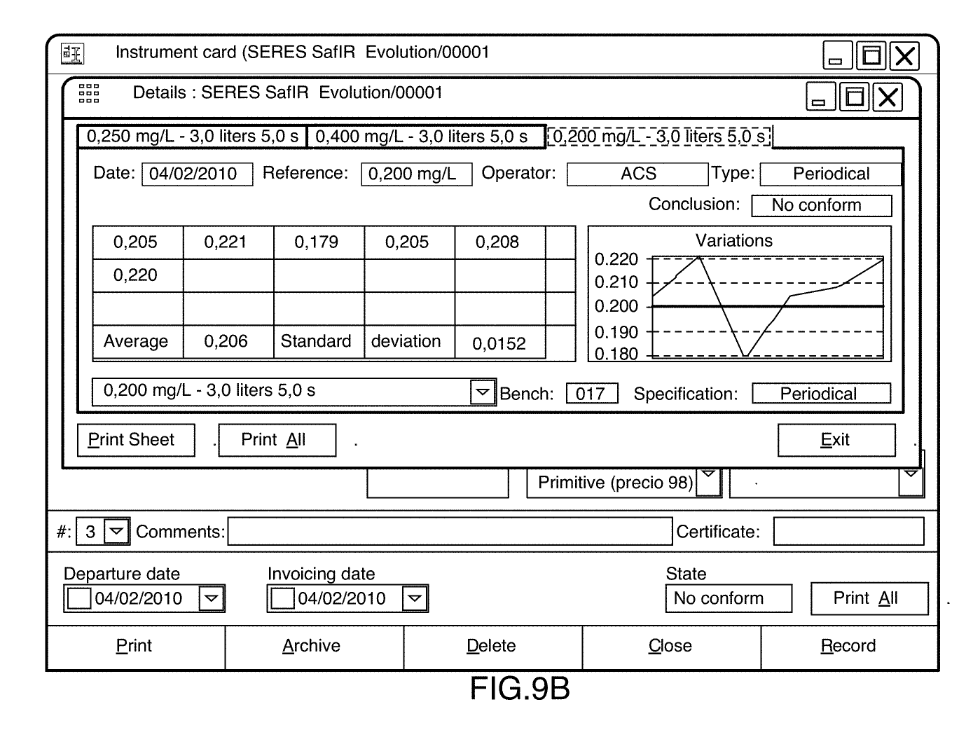

It allows the setting of different parameters (cycles, volume, flow, breath profiles, concentrations, . . . ) and continuously monitors them on graphs. It also ensures the graphical and mathematical analysis of different data (regression calculus, standard deviation, results estimation, stability, calibration coefficient evaluation, etc). Examples of the above are shown in FIG. 9.

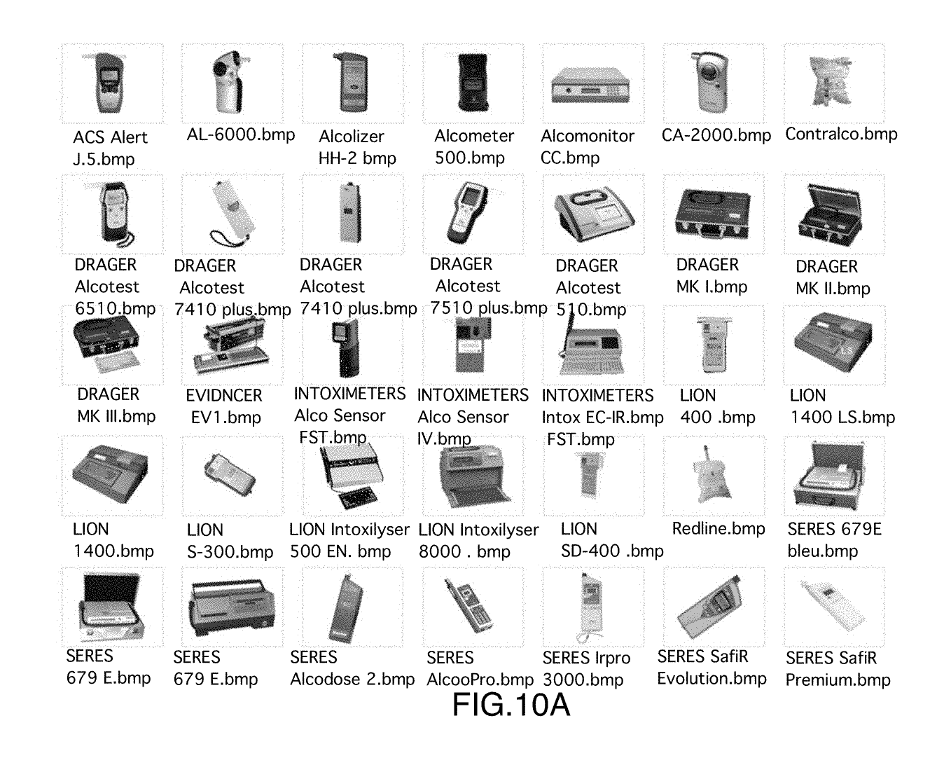

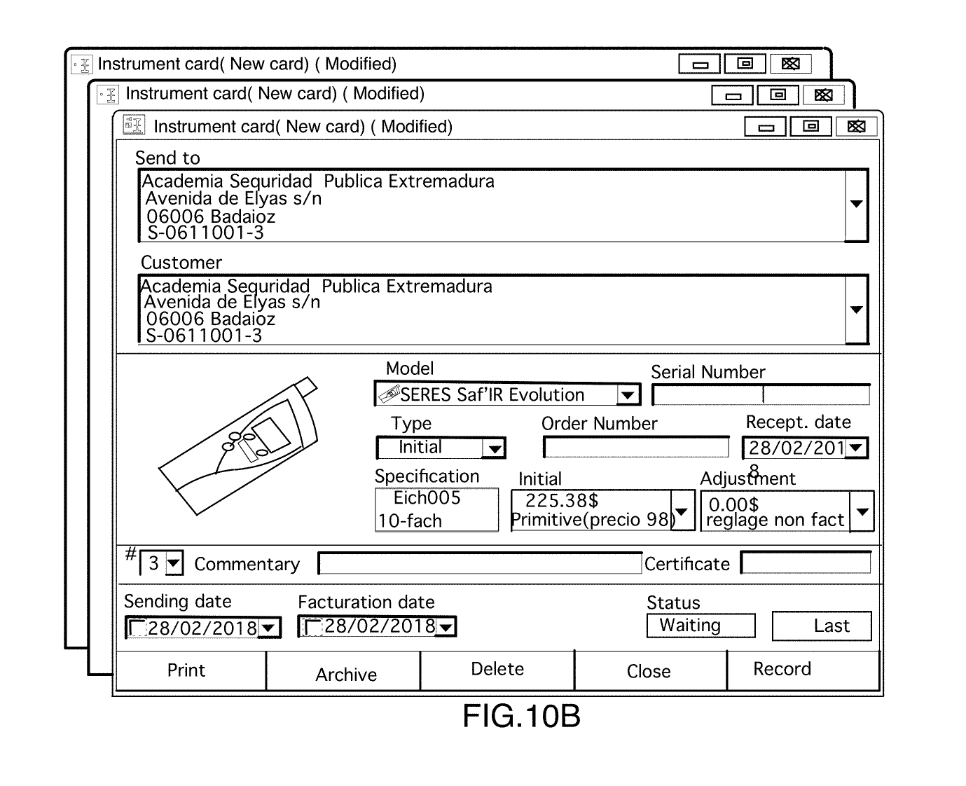

Additional software can manage the instruments from their receiving to the report of calibration. It also permits to do some programs of confirming and to analyze the cell's behavior. Several filling functions take into account all the results (apparatus values, calibration values, bench values, . . . ) and statistical functions look at the changes. Examples of the above are shown in FIG. 10.

### Principle of Sampling and Gas Generation

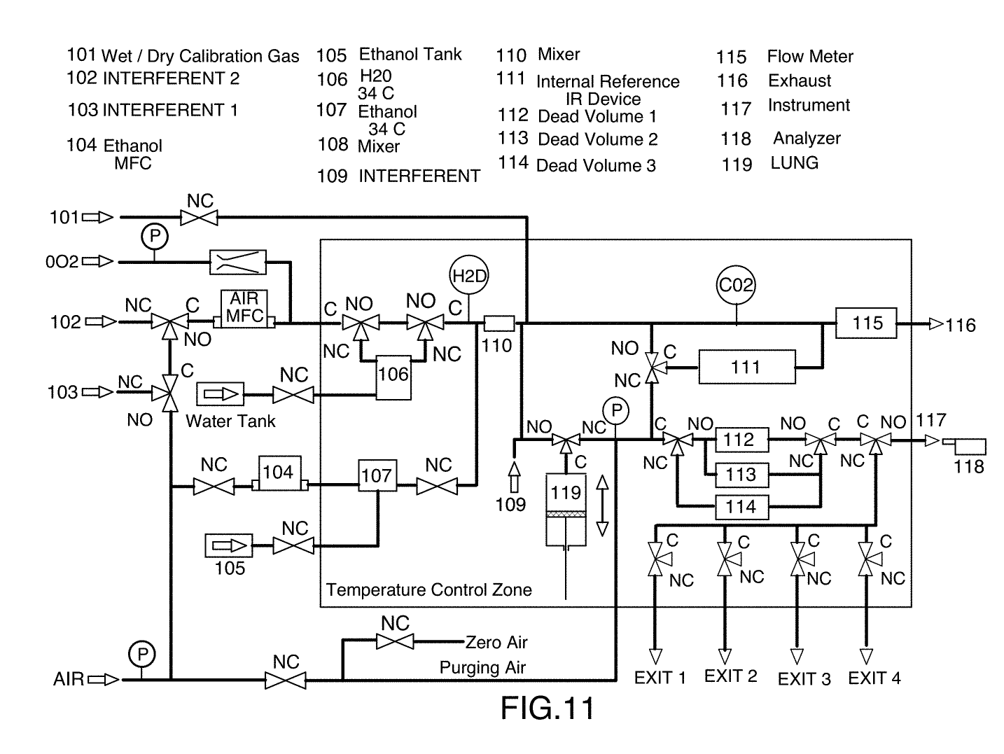

The operation of the bench will hereinafter be described with reference to FIGS. 11-17.

FIG. 11 is a schematic of the flow circuitry.

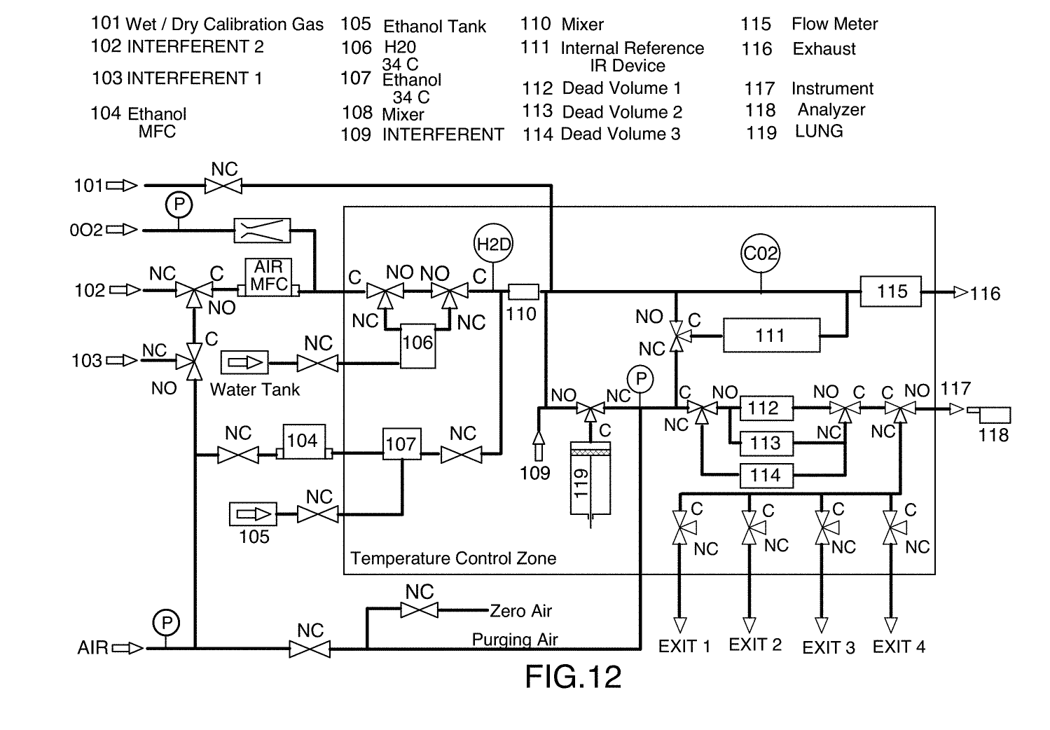

FIG. 12 shows a standby mode.

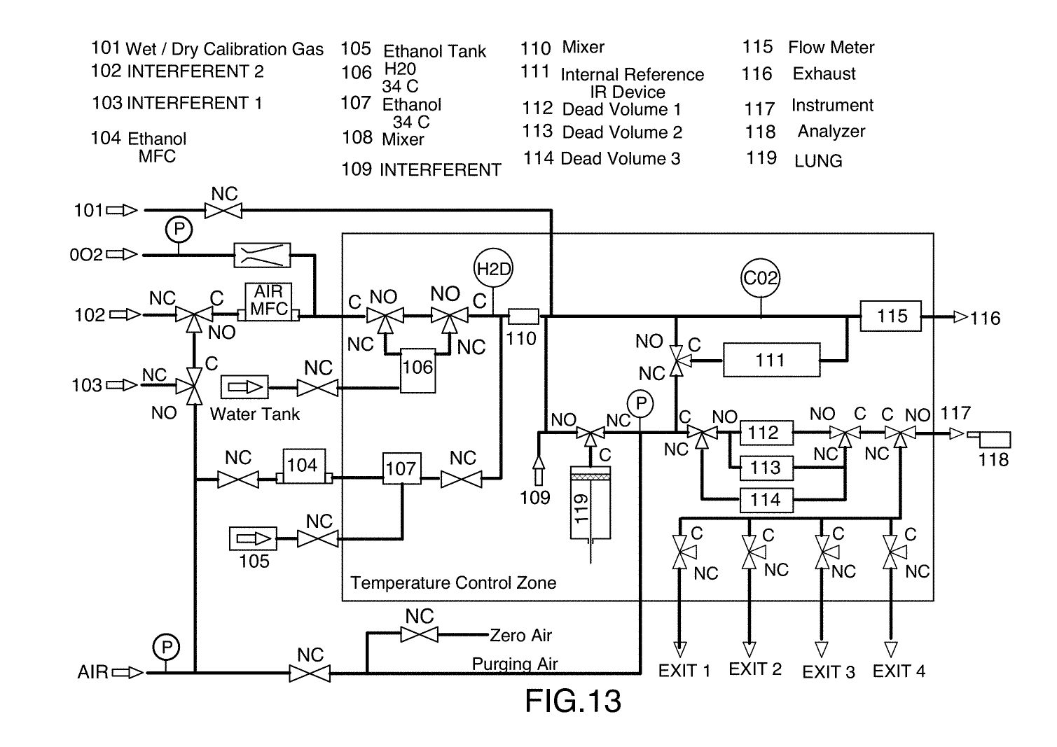

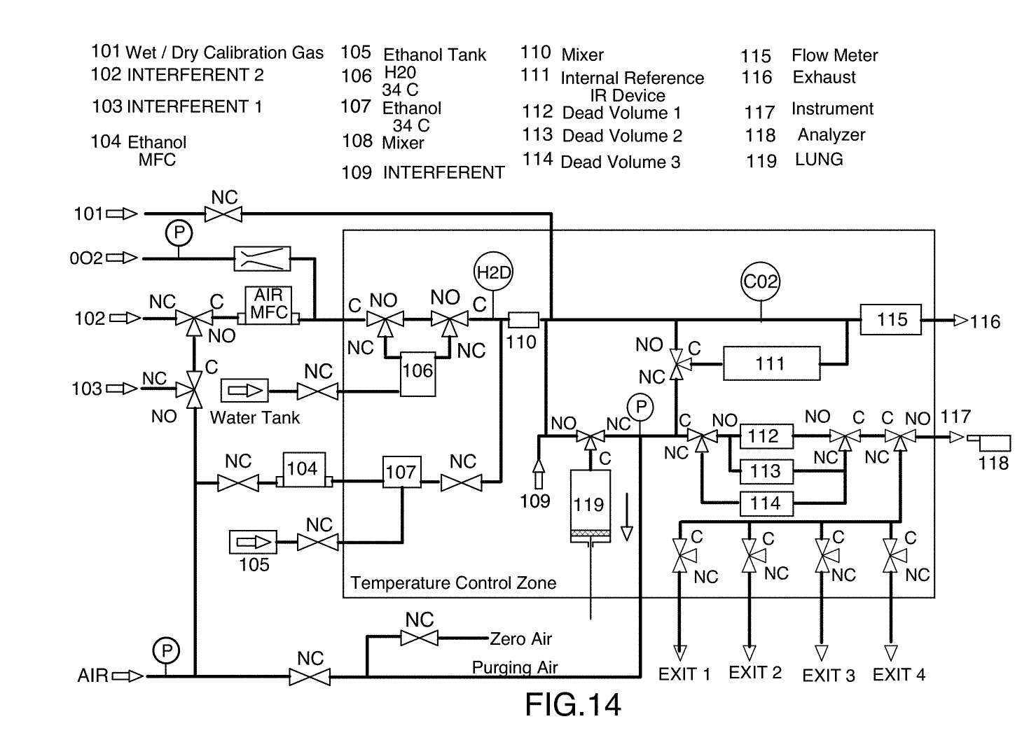

FIGS. 13 and 14 show the steps associated with breath preparation.

Mixture generation is shown in FIG. 13. After air is introduced into the system, it goes through two mass flow controls (MFCs); one for the ethanol tank and the other for the water tank. Each MFC accurately controls the amount of air that goes into each tank. After the air leaves the MFCs, it will go through the tanks, which are heated at 34*C. The air from the ethanol tank and the water tank will then mix together. A three-way valve system attaches to the water tank and allows the air to either pass through the tank to become moist air or to bypass the tank to stay at dry air. In the case of CO₂, the gas will go through another MFC and mix with the air which leaves the water MFC and proceed through the rest of the system as normal. After the gases are mixed, they will proceed until they reach the exhaust system.

Sampling is shown in FIG. 14. The artificial lung was designed to contain the piston, which is driven by the electrical actuator that can precisely move it up and down with sensors, and the cylinder. Sampling occurs when the piston moves down and, therefore, draws the mixture of gases from the mixture line and stores it inside the lung.

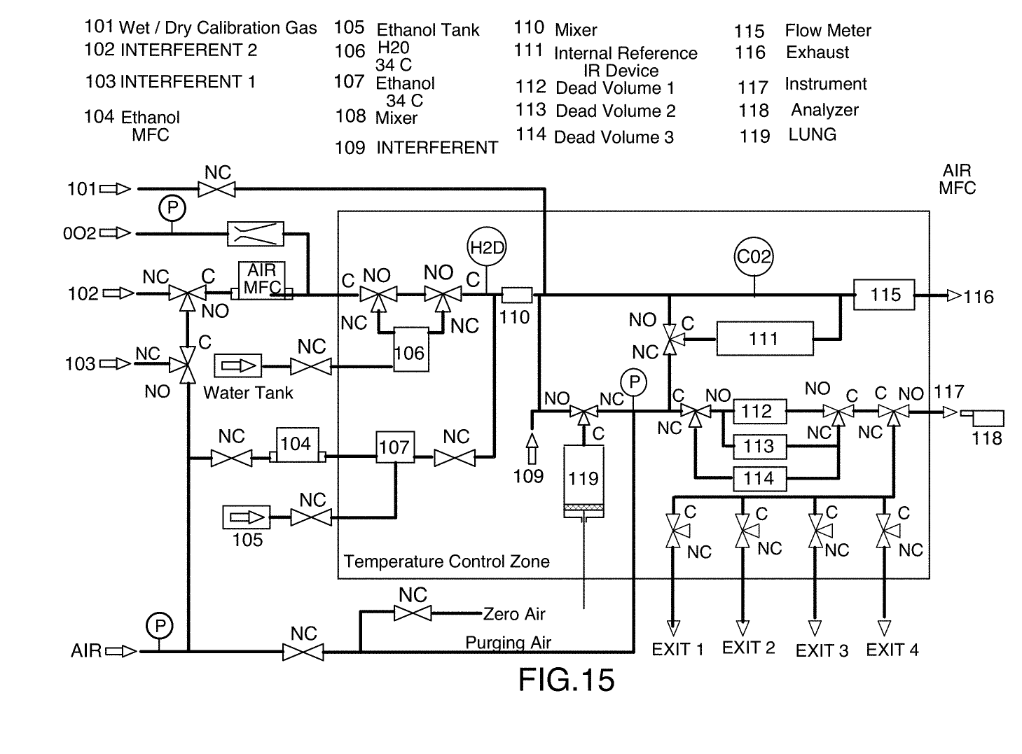

FIG. 15 shows the circuitry waiting for breath.

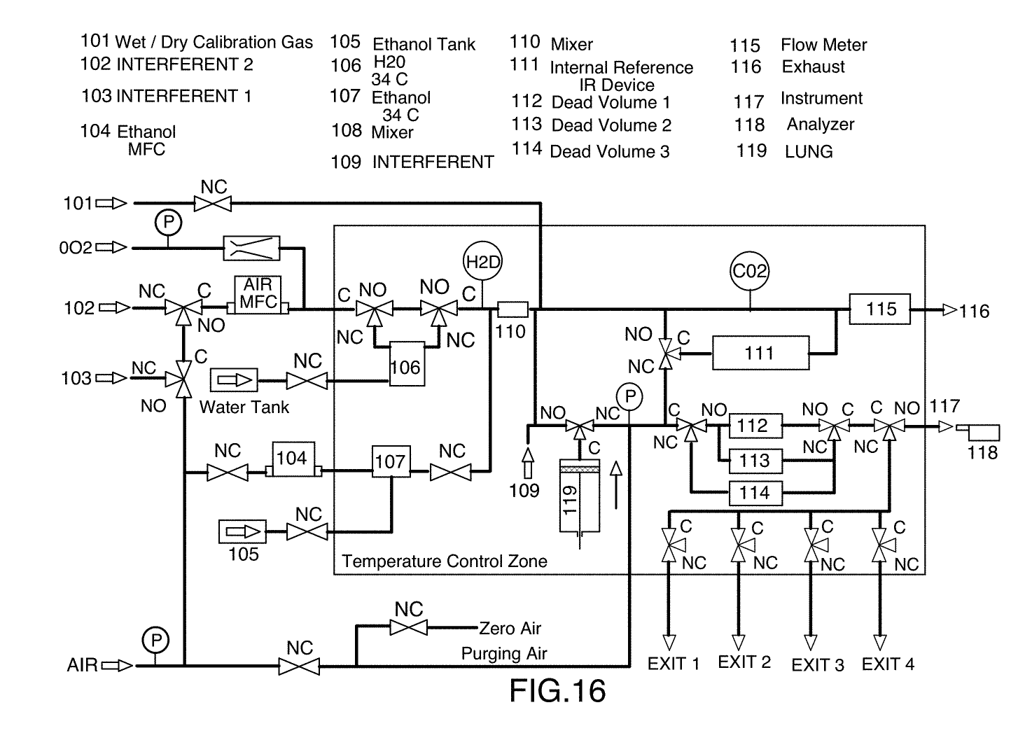

FIG. 16 shows the circuitry generating a breath. After the valve opens, the piston moves up and causes the sampling to move to the testing line. The movement of the piston will precisely be controlled in order to simulate the human breath profile. Before the sample goes to the unit. it will go through the dead air volume system in order to simulate the upper respiratory tract of a human lung. After each test, the sampling line will be purged by the zero air so that it can clean what is left over and remove the old sampling.

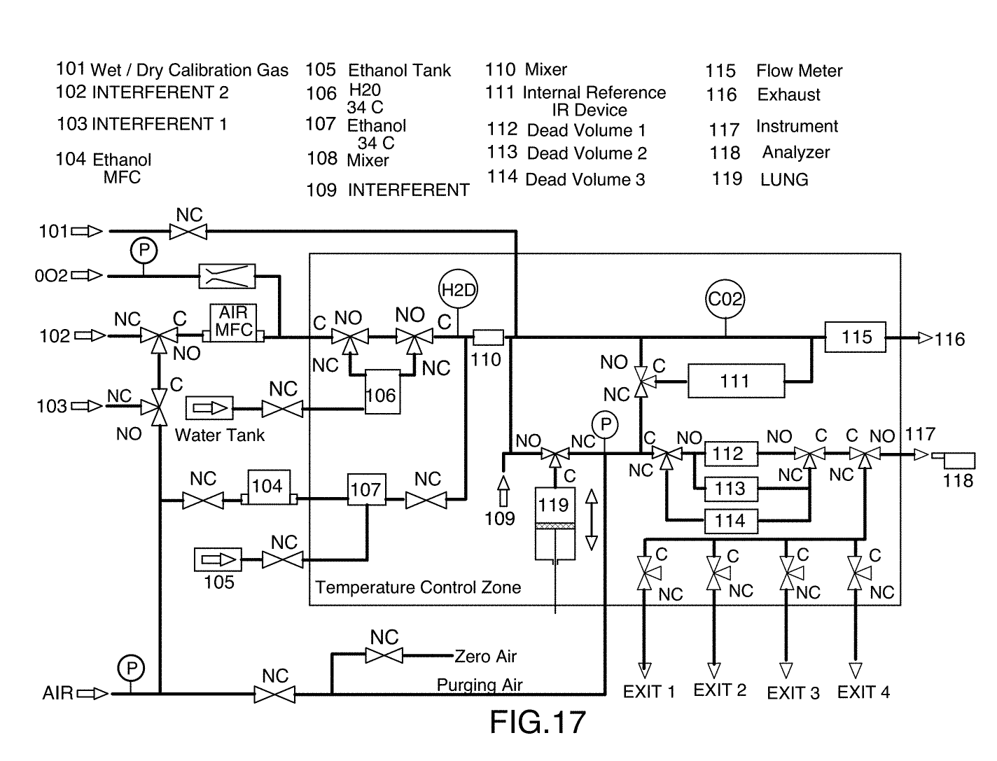

When the test bench needs to do the calibration, different concentrations of calibration gas will be introduced to the calibration line. After they are introduced, the pump of the IR system will turn on and move the calibration gas to the IR unit in order to calibrate. FIG. 17 is illustrative in this regard.

### Bench and Options Characteristics

Persons of ordinary skill will readily recognize that the bench provides great advantage in terms of flexibility in operation:

BT&EBA Calibration & Verification Bench
Concentration: 0 to 2.500 mg/l (step of 0.001 mg/l)
Blow device: Artificial lung
Flow: 0 to 80 L/min
Breath time: 0 to 30 s
Breath temperature: 34° C. PID control with external flexible thermostatic tube
Bench body temperature: 35.5° C.
Uncertainty: Less than 1.25% (or 5 μg/L)
Breath volume: 0 to 5 L/min
Integrated electronic interface (front side of the bench)

### CO₂ Option

Manage a CO₂ concentration from 0 to 10%

### Dead Volume Option

3 s “plateau” (small dead volume) or 1.5 s “plateau” (big dead volume) selected by the three-way valve
Simulates alcohol in the upper respiratory tract

### Dry/Wet Gas Option: Generation of Dry Gas Mode

<0.5% RH (required dryer for compressed air) or wet gas mode (>95% RH)

### Human Breath Option

Flow profile meets with OIML R126 2012(E)
Constant, varied or designed by the user
Simulates alcohol in the upper respiratory tract
OIML R126 2012 11.4.4.2 Influence factors of the conditions of injection
   Influence of delivered volume and exhalation duration
   Influence of flow rate and injection duration
   Influence of variations in the flow rate during exhalation
   Influence of duration of the plateau during injection
   Influence of an interruption in the breath flow

### Ethanol Monitoring Option

Monitoring of ethanol generated

### External Reference

Evidential breath analyzer can interface connection to the bench

### Metrological Monitoring Option

Sensor for ethanol (curve of the breath), CO₂ concentration
Sensor for humidity, pressure, breath temperature, breath flow

### Integrated Dry Calibration Device

Connection of six to nine cylinders
Vacuum pump

### External Cylinders Interferent Option

Connection for two external cylinders (example mixture with air & acetone and air & CO) to replace carrier gas (air)

### Multiplexer Device Option

Possibility to connect up to 4 devices for automatic calibration and controls (devices must be equipped with an RS232 or USB interface)

### Software

Under Windows 7 (language English) and C++ for PLC.

### Multiplexer

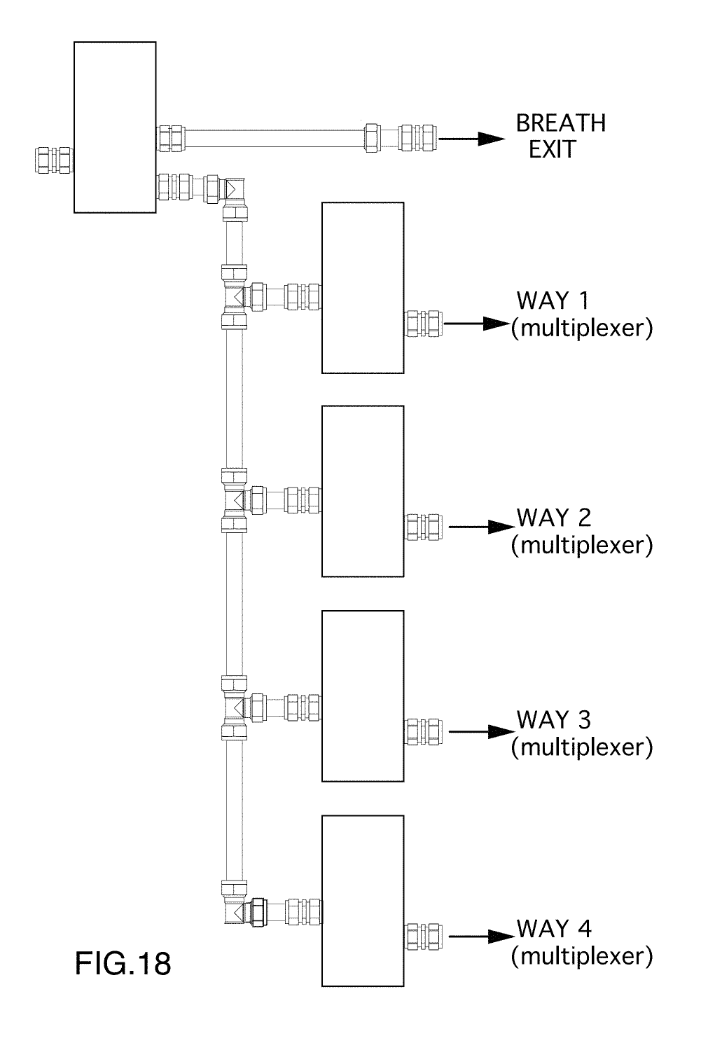

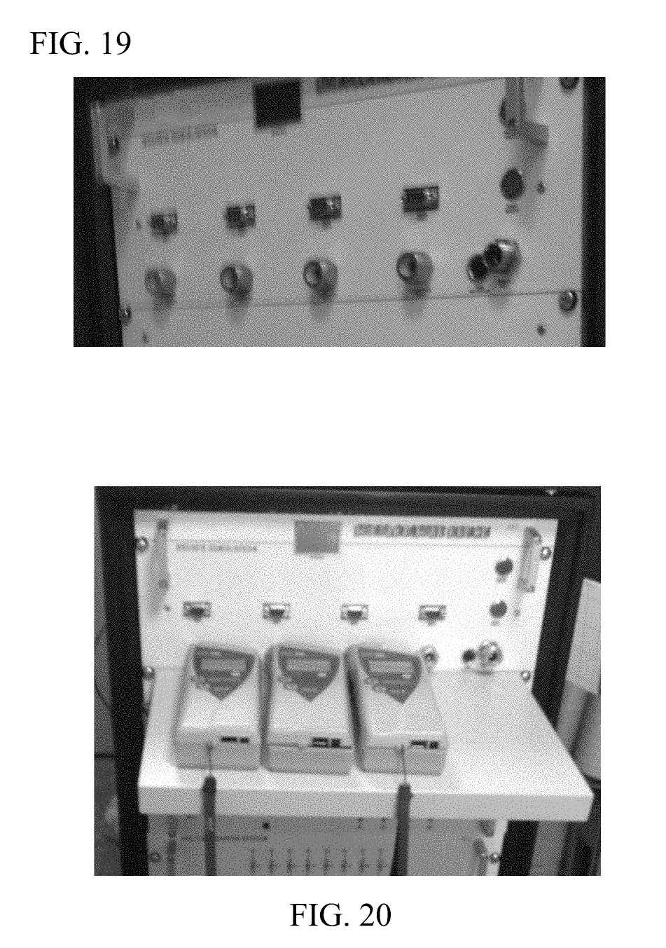

The multiplexer is a system, shown schematically in FIG. 18, of four additional output ports (selected with three-way valves) that allow four verifications/calibrations to be performed at the same time, when instruments are equipped with serial communication. “At the same time” does not mean the breath is simultaneously done on 4 units (for example, in order to do a 3 L breath simultaneously, the bench would need a 12 L lung which is not the case). “At the same time” means that when it is possible to control the instrument with a serial connection, a complete cycle of initial (or periodical or customized) verification will be automatically done by the bench, and the results will be recorded and eventually printed. FIG. 19 shows the panel that is adapted to receive four units, and FIG. 20 shows three units operatively coupled to the bench for calibration.

### Standards

The advised quality and range for standard are:
Hydroalcoholic solution: Range: 0 to 2.0 g/l, Certified uncertainties: 0.1%, Conditioning: bottle of 1 or 5 liters
Dry gases: Range: 0 to 1100 ppm, Certified uncertainty: at least 2% (2 ppm below 100 ppm), Conditioning: cylinders of 20 or 50 liters

Whereas a specific embodiment is herein shown and described, variations are possible. Accordingly, the invention should be understood to be limited only by the claims, purposively construed.

What is claimed is:

1. A method for calibrating a breath alcohol tester, the method comprising:
delivering to the tester a sample of gas that varies in terms of ethanol concentration, CO₂ concentration, flow rate, pressure and temperature in a manner equivalent to the variances exhibited by a human.

2. Apparatus adapted to carry out the delivery of claim 1.
